(19) United States
(12) Patent Application Publication
(10) Pub. No.: US 2025/0073317 A1
(43) Pub. Date: Mar. 6, 2025

(54) CHOLINE ACETYLTRANSFERASE AS A THERAPY FOR ENDOTOXEMIA, SEPSIS, COLITIS AND INFLAMMATORY DISEASES

(71) Applicant: The Feinstein Institutes for Medical Research, Manhasset, NY (US)

(72) Inventors: Sangeeta S. CHAVAN, Syosset, NY (US); Kevin J. TRACEY, Old Greenwich, CT (US)

(73) Assignee: The Feinstein Institutes for Medical Research, Manhasset, NY (US)

(21) Appl. No.: 18/293,972

(22) PCT Filed: Aug. 25, 2022
(86) PCT No.: PCT/US2022/041484

§ 371 Date: Jan. 31, 2024

Related U.S. Application Data
(60) Provisional application No. 63/239,050, filed on Aug. 31, 2021.

(51) Int. Cl. A61K 38/45; A61K 31/14; A61K 31/27; A61K 31/445; A61K 31/55; A61K 31/7076; A61K 45/06; A61K 47/60; A61P 1/00; A61P 29/00; A61P 31/00

(52) U.S. Cl. CPC A61K 38/45; A61K 31/14; A61K 31/27; A61K 31/445; A61K 31/55; A61K 31/7076; A61K 45/06; A61K 47/60; A61P 1/00; A61P 29/00; A61P 31/00; C12Y 203/01006

(57) ABSTRACT

This invention provides for methods of treating one or more of endotoxemia, sepsis, colitis, elevated cytokine levels and an inflammatory condition in a subject in need thereof, which comprises administering an effective amount of a composition comprising purified choline acetyl transferase (ChAT) or purified ChAT conjugated to polyethylene glycol (PEGylated ChAT) and a pharmaceutically acceptable carrier to said subject.

### CROSS-REFERENCE TO RELATED APPLICATIONS

This application claims the benefit of U.S. Provisional Patent Application No. 63/239,050 filed on Aug. 31, 2021, the contents of which are herein incorporated by reference into the subject application.

### FIELD OF THE INVENTION

This invention provides for methods and compositions for attenuating endotoxemia, sepsis, colitis and inflammatory diseases by administering compositions comprising choline acetyl transferase (“ChAT”) and/or ChAT that is conjugated to polyethylene glycol (“PEGylated ChAT” or “PEG-ChAT”).

### BACKGROUND OF THE INVENTION

During sterile injury, ischemia, or infection, the activation of immune responses protects against detritus of tissue injury and initiates wound healing and repair mechanisms. These responses are normally short-lived, and the resolution of inflammation is an essential component of immunological homeostasis. Abundant evidence further indicates that nonresolution of inflammation drives the pathogenesis of a wide range of inflammatory diseases. Examples of these disease states include autoimmune diseases, pancreatitis, cytokine storm, ischemia reperfusion injury, sepsis, arthritis, myocarditis, inflammatory bowel disease, atherosclerosis, obesity, cancer, pulmonary disease, neurodegeneration, multiple sclerosis, and rheumatoid arthritis.

ChAT is an enzyme involved in the final step in the synthesis of acetylcholine (“ACh”) in humans and non-human animals. The enzyme catalyzes the transfer of an acetyl group from the coenzyme, acetyl-Coenzyme-A, to choline yielding acetylcholine. ACh has an important role in the body where it functions as a neurotransmitter in the cholinergic systems in the brains and bodies of many animals, including humans. Hence, the modulation of levels of ChAT may lead to valuable ways to treat many disease states. U.S. Pat. No. 6,656,743 B2 provides for the isolated ChAT protein and methods to treat degenerative disorders, such as Alzheimer's Disease and ALS, or Parkinson's Disease.

Studies have shown that ChAT is useful for improving cognitive disorders. The administration of ChAT coupled to protein transduction domain (PTD) improved age-related cognitive defects in aged mice (Fu et al., “Complimentary remedy of age-related learning and memory deficits via exogeneous choline acetyltransferase.” Biochem. Biophys. Res. Commun., 336 (1), 268-73 (2005)). Similarly, U.S. Pat. No. 9,248,152 showed that implantation of neural stem cells (“NSCs”) that stably express human ChAT improved learning and memory in a rat model and proposed the use of these stem cells for treating Alzheimer's disease as well as cognitive disorders due to other brain diseases and aging. WO 2005/071071 proposes using a PTD-ChAT fusion protein for treating neurodegeneration disease, preventing Alzheimer's disease, and advancing memory of learning. Similarly, U.S. Pat. No. 7,083,930 B2 proposes that ChAT polypeptides may be used to treat amyotrophic lateral sclerosis, Alzheimer's Disease, senile-dementia, multi-infarct dementia, familial disautonomia, Huntington's Disease, mental retardation, memory loss, and myasthenia gravis, as well as disorders known to involve the cholinergic system or affect its pathways and nerves in the body, including for example gut and GI disorders, cord disorders, including movement, continence and sensation, stem disorders, including sleep, blood pressure, respiration, and balance, hypothalamus disorders, including temperature, respiration, and endocrine function, and limbic system disorders, including schizophrenia, memory disorders, and dementia.

There have also been reports in the literature that administering NSCs expressing ChAT and neurotrophins attenuate brain and capillary injuries and restore neurobehavioral function via neuroprotection and anti-inflammatory activities (Kim et al., “Neuroprotective effects of human neural stem cells over-expressing choline acetyltransferase in a middle cerebra artery occlusion model,” J. Chem. Neuroanat., 103, 101730 (2020). Further, it has been reported that neuronal and non-neuronal ACh provide anti-inflammatory benefits (Han et al., “The cholinergic anti-inflammatory pathway: An innovative treatment strategy for neurological diseases,” Neusci. Biobehav. Rev., 77, 358-368 (2017)). Sales is a review that analyzes the participation of the cholinergic system in inflammation and discusses the role of different muscarinic and nicotinic drugs that are used to treat skin inflammatory disorders, asthma, and chronic obstructive pulmonary disease as well as intestinal inflammation and systemic inflammatory diseases (Sales, Antiinflamm. Antiallergy Agents Med. Chem., 23 (2), 109-116 (2013)).

WO 2020/123611 provides for compositions comprising ChAT and PEGylated ChAT for treating hypertension.

There are a number of inflammatory diseases that are not responsive to current anti-inflammatory therapeutics. Further, there is a continuing need for a therapeutic that is simple to administer. These and other objectives are achieved by the present invention discussed below.

The disclosures of all publications, patents and patent applications mentioned herein are hereby incorporated by reference in their entirety into the subject application to more fully describe the art to which the subject invention pertains. However, the citation or identification of any document in this application is not an admission that such document is available as prior art to the present invention.

### SUMMARY OF THE INVENTION

The present invention provides for methods and compositions for attenuating and treating endotoxemia, sepsis, colitis and inflammatory conditions by administering compositions comprising purified choline acetyl transferase (“ChAT”) or purified ChAT that is conjugated to polyethylene glycol (“PEGylated ChAT”). This invention further provides for methods for reducing cytokine levels in a subject in need thereof as well as compositions that achieve this effect. Further, this invention provides for methods and compositions for treating or reducing symptoms associated with COVID-19 in a subject in need thereof.

It is noted that in this disclosure and particularly in the claims and/or paragraphs, terms such as “comprises”, “comprised”, “comprising”, and the like can have the meaning attributed to it in U.S. Patent law: e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

FIG. 1 demonstrates that administration of ChAT protein attenuates inflammation in response to endotoxin challenge. Error bars represent ±SEM, * p<0.05.

FIG. 2 demonstrates that administration of ChAT protein attenuates inflammation in response to endotoxin challenge in a dose-dependent manner. Error bars represent ±SEM, * p<0.05.

FIG. 3 demonstrates that administration of PEG-ChAT protein attenuates inflammation in response to endotoxin challenge. Error bars represent ±SEM, ** p<0.01.

FIG. 4A-4B demonstrates that administration of anti-ChAT antibody exacerbated TNF production in acute endotoxemia. Mice were administered Vehicle or 1 mg/kg anti-ChAT antibody (17E) 30 minutes prior to lipopolysaccharide (LPS) injection. Serum and spleen were collected 90 minutes after endotoxin administration and TNFα levels were measured by ELISA. In both serum (A) and spleen (B), antibody treated mice showed even more elevated levels of TNF compared to the vehicle group. Analyses are shown as mean±SEM. Mann-Whitney test, p=0.015 (serum), p=0.016 (spleen). Error bars represent±SEM, * p<0.05.

FIG. 5 demonstrates that administration of ChAT protein improves survival in a lethal sepsis model. ChAT (1 mg/kg) was injected once per day on Days 0-5.

FIG. 6 demonstrates that administration of PEG-ChAT protein improves survival in a lethal sepsis model. PEG-ChAT was injected once per day on Days 0-5.

FIG. 7 demonstrate that administration of PEG-ChAT protein improves survival in a lethal sepsis model. Administration of PEG-ChAT improved survival in a cecal ligation and puncture (CLP) model. Wild type mice were subjected to lethal CLP. All mice were injected with vehicle or 1 mg/kg PEG-ChAT 30 min before the CLP procedure, and twice daily after for 7 days. PEG-ChAT administered mice demonstrated higher survival rate compared to vehicle treated group. **Mantel-Cox test p=0.0157.

FIG. 8 demonstrates that administration of ChAT or PEG-ChAT suppresses cytokine storm in response to SARS COV2-spike protein challenge. IL-6 (a pro-inflammatory cytokine) levels are illustrative of cytokine storm.

FIG. 9A-9B demonstrate that the administration of ChAT or PEG-ChAT attenuates decrease in body weight (A) and % change in body weight (B) in a preclinical DSS model of colitis. Dose of ChAT or PEG-ChAT=1 mg/kg, 2 times per day for 7 days. * p<0.05; ** p<0.01.

FIG. 10 demonstrates that the administration of ChAT or PEG-ChAT improves disease activity index in a preclinical DSS model of colitis.

FIG. 11 demonstrate that the administration of ChAT or PEG-ChAT improves colon length in a preclinical DSS model of colitis. ** p<0.01; **** p<0.0001.

FIG. 12 demonstrates that the administration of ChAT or PEG-ChAT significantly decreases circulating levels of TNF in a preclinical model of colitis, an inflammatory condition. Error bars represent ±SEM.

FIG. 13 demonstrates that the administration of ChAT or PEG-ChAT decreases circulating levels of TNF over time in a preclinical DSS model of colitis, an inflammatory condition. Error bars represent ±SEM, * p<0.05.

FIG. 14 demonstrates that administration of Dexamethasone plus ChAT or PEG-ChAT reduces inflammation in acute endotoxemia. Error bars represent ±SEM, * p<0.05; ** p<0.01.

The following detailed description, given by way of example, but not intended to limit the invention solely to the specific embodiments described, may best be understood in conjunction with the accompanying drawings.

### DETAILED DESCRIPTION OF THE INVENTION

An embodiment of this invention provides for a method of treating one or more of endotoxemia, sepsis, colitis and an inflammatory disease or condition in a subject in need thereof, which comprises administrating (e.g., by injecting or infusing intravenously or intraperitoneally, or by administering orally or intranasally or intra-rectally) an effective amount of a composition comprising ChAT or PEGylated ChAT and a pharmaceutically acceptable carrier to said subject.

Another embodiment of this invention provides for a method for reducing circulating pro-inflammatory cytokine levels in a subject in need thereof, which comprises administering (e.g., by injecting or infusing intravenously or intraperitoneally, or by administering orally or intranasally or intra-rectally) an effective amount of a composition comprising ChAT or PEGylated ChAT and a pharmaceutically acceptable carrier to said subject.

Another embodiment of this invention provides for a method for treating or reducing symptoms associated with COVID-19 in a subject in need thereof, which comprises administering (e.g., by injecting or infusing intravenously or intraperitoneally, or by administering orally or intranasally or intra-rectally) an effective amount of a composition comprising ChAT or PEGylated ChAT and a pharmaceutically acceptable carrier to said subject.

Another embodiment provides purified choline acetyl transferase (ChAT) and/or purified ChAT conjugated to polyethylene glycol (PEGylated ChAT or PEG-ChAT) for use in a method of treatment of one or more of endotoxemia, sepsis, colitis and an inflammatory condition.

Another embodiment provides purified choline acetyl transferase (ChAT) and/or purified ChAT conjugated to polyethylene glycol (PEGylated ChAT or PEG-ChAT) for use in a method of reducing circulating cytokine levels in a subject.

Another embodiment provides purified choline acetyl transferase (ChAT) and/or purified ChAT conjugated to polyethylene glycol (PEGylated ChAT or PEG-ChAT) for use in a method of treating or reducing symptoms associated with COVID-19.

In some of the above embodiments the subject is a human. In another embodiment, the subject is a non-human animal, such as for example, mammals, such as felines (e.g., cats), canines (e.g., dogs), equines (e.g., horses, donkeys, or zebras), llamas, porcines, and bovines, and birds (e.g., chickens).

In the above-provided methods, the symptoms associated with inflammation are treated or diminished.

Inflammatory conditions treated or attenuated by reducing circulating cytokine levels in a subject in need thereof include appendicitis, peptic, gastric or duodenal ulcers, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alveolitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, atherosclerosis, thrombophlebitis, pericarditis, myocarditis, myocardial ischemia, periarteritis nodosa, rheumatic fever, coeliac disease, congestive heart failure, adult respiratory distress syndrome, Alzheimer's disease, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, Goodpasture's syndrome, Behcet's syndrome, allograft rejection, graft-versus-host disease, ankylosing spondylitis, Type I diabetes, ankylosing spondylitis, Berger's disease, reactive arthritis (Reiter's syndrome) or Hodgkin's disease. In more preferred embodiments, the condition is appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection or graft-versus-host disease.

In some embodiments, the inflammatory conditions are appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, coeliac disease, congestive heart failure, adult respiratory distress syndrome, cerebral infarction, cerebral embolism, spinal cord injury, paralysis, allograft rejection or graft-versus-host disease.

In other embodiments, the inflammatory conditions treated by the methods provided for herein include myocarditis, pancreatitis, inflammatory bowel disease, colitis, peritonitis, ulcerative pseudomembranous, acute or ischemic colitis, diverticulitis, epiglottis, endotoxemia, rheumatoid arthritis, osteoarthritis, asthma, or ischemia reperfusion injury. In yet other embodiments, the inflammatory conditions are peptic, gastric, or duodenal ulcers. In further embodiments, is endotoxemia, colitis or myocarditis. In yet other embodiments, the inflammatory condition is due to chemotherapy-induced toxicity or radiation-induced toxicity.

Further, in the above embodiments, the composition is injected intravenously and consists essentially of or consists of ChAT and a pharmaceutically acceptable carrier. Alternatively, the composition is injected intravenously and consists essentially of or consists of PEGylated ChAT. Preferably, the ChAT is human ChAT. The ChAT can be a recombinant ChAT. In one embodiment, the ChAT is ChAT isoform R.

The term “ChAT” refers to recombinant ChAT in which other proteins, such as neurotrophins and endotoxins, and other contaminants have been removed through techniques such dialysis, chromatography, filtration, etc. Typically, these techniques produce a ChAT protein that is 99-99.9% pure.

The ChAT can be conjugated to polyethylene glycol (PEG). For example, ChAT can be conjugated to 2-24 PEG chains. In one embodiment, ChAT is conjugated to 12 PEG chains. Each PEG chain can have a molecular weight of, for example, 200-2,000 daltons. In one embodiment, PEG-ChAT has a molecular weight about 20,000 daltons greater than ChAT. Conjugation of PEG to ChAT can be effective to increase the solubility and/or half-life of ChAT.

ChAT or PEGylated ChAT can be administered by any route known to those skilled in the art. In different embodiments, ChAT or PEGylated ChAT is administered by intravenous or intraperitoneal injection or infusion via a device such as an osmotic pump. In one embodiment, ChAT or PEGylated ChAT is administered by serial injections or infusions spaced over an interval of one or more days.

Also provided is a pharmacological composition comprising choline acetyltransferase (ChAT) conjugated to polyethylene glycol (PEG) and a pharmaceutically acceptable carrier. For example, ChAT can be conjugated to about 2 to about 24 PEG chains. In one embodiment, ChAT is conjugated to 12 PEG chains. Each PEG chain can have a molecular weight of, for example, about 200 to about 2,000 daltons. In one embodiment, PEG-ChAT has a molecular weight about 20,000 daltons greater than ChAT.

Examples of acceptable pharmaceutically acceptable carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution.

In the above-described embodiments, the compositions may further contain at least one additional anti-inflammatory agent (e.g., a non-steroidal anti-inflammatory agent or a steroid) and/or acetylcholine esterase inhibitor. Non-limiting examples of non-steroidal anti-inflammatory agents include aspirin, indomethacin, naproxen sodium, COX-2 inhibitors, such as celecoxib, diclofenac, diflunisal, etodolac, ketoprofen, ibuprofen, acetaminophen, fenoprofen, meloxicam, piroxicam, tolmetin, sulindac, phenylbutazone, and oxyphenbutazone. Non-limiting examples of steroids include prednisone, triamcinolone, hydrocortisone, beclomethasone, betamethasone, cortisone, or dexamethasone, etc.

The composition can also contain other agents that may enhance the enzyme activity of ChAT such as choline, any form of choline that can serve as the substrate, acetyl-coA or acetylcholinesterase inhibitors. Non-limiting examples of acetylcholine esterase inhibitors include neostigmine, donepezil, rivastigmine, galantamine, phenserine, huperzine A, and BW284c51. In some embodiments, the above-described compositions comprise choline, acetyl coenzyme A, or both.

### Modes of Administration

The compositions of the present invention may be administered by any conventional means such as oral, intravenous, intraperitoneal, intranasal, or intra-rectal (enema) administration. In one embodiment, the mode of administration is intravenous or intraperitoneal. In another embodiment, the mode of administration is intranasal. In another embodiment, the mode of administration is intra-rectal. Non-limiting examples of intra-rectal administration include a suppository, a micro-enema, a large volume enema, using a specialized catheter designed for repetitive rectal administration of medications.

The compositions of the present invention may be administered once, twice, thrice, or four times daily depending upon the subject, condition, and the disease state. The determination of a specific dose for a specific disease state is typically well within the skill level of the skilled artisan.

In an embodiment, human choline acetyltransferase has the following amino acid sequence (GenBank: AAA14245.1; SEQ ID NO: 1):

| | |
|---|---|
| 1 | mglrtakkrg lggggkwkre egggtrgrre vrpacflqsg grgdpgdvgg pagnpgcsph |
| 61 | praatrpppl pahtpahtpe wcgaasaeaa eprragphlc ipapgltktp ilekvprkma |
| 121 | aktpsseesg lpklpvpplq qtlatylqcm rhlvseeqfr ksqaivqqfg apgglgetlq |
| 181 | qkllerqekt anwvseywln dmylnnrlal pvnsspavif arqhfpgtdd qlrfaaslis |
| 241 | gvlsykalld shsiptdcak pelsgqplcm kqyyglfssy rlpghtqdtl vaqnssimpe |
| 301 | pehvivaccn qffvldvvin frrlsegdlf tqlrkivkma snederlppi glltsdgrse |
| 361 | waeartvlvk dstnrdsldm ierciclvcl dgpggvelsd thralqllhg ggysknganr |
| 421 | wydkslqfvv grdatcgvvc ehspfdgivl vqctehllkh mtqssrklir adsvselpap |
| 481 | rrlrwkcspe iqghlassae klqrivknld fivykfdnyg ktfikkqkcs pdafiqvalq |
| 541 | lafyrlhrrl vptyesasir rfqegrvdni rsatpealaf vravtdhkaa vpasekllll |
| 601 | kdairaqtay tvmaitgmai dnhllalrel aramckelpe mfmdetylms nrfvlstsqv |
| 661 | ptttemfccy gpvvpngyga cynpqpetil fcissfhsck etssskfaka veeslidmrd |
| 721 | lcsllpptes kplatkekat rpsqghqp. |

In an embodiment, human choline acetyltransferase isoform R has the following amino acid sequence (GenBank: AAK08955.1; SEQ ID NO:2):

| | |
|---|---|
| 1 | maaktpssee sglpklpvpp lqqtlatylq cmrhlvseeq frksqaivqq fgapgglget |
| 61 | lqqkllerqe ktanwvseyw lndmylnnrl alpvnsspav ifarqhfpgt ddqlrfaasl |
| 121 | isgvlsykal ldshsiptdc akgqlsgqpl cmkqyyglfs syrlpghtqd tlvaqnssim |
| 181 | pepehvivac cnqffvldvv infrrlsegd lftqlrkivk masnederlp piglltsdgr |
| 241 | sewaeartvl vkdstnrdsl dmierciclv cldapggvel sdthralqll hgggysknga |
| 301 | nrwydkslqf vvgrdgtcgv vcehspfdgi vlvqctehll khmtqssrkl iradsvselp |
| 361 | aprrlrwkcs peiqghlass aeklqrivkn ldfivykfdn ygktfikkqk cspdafiqva |
| 421 | lqlafyrlhr rlvptyesas irrfqegrvd nirsatpeal afvravtdhk aavpasekll |
| 481 | llkdairaqt aytvmaitgm aidnhllalr elaramckel pemfmdetyl msnrfvlsts |
| 541 | qvptttemfc cygpvvpngy gacynpqpet ilfcissfhs cketssskfa kaveeslidm |
| 601 | rdlcsllppt eskplatkek atrpsqghqp |

“And/or” as used herein, for example, with option A and/or option B, encompasses the separate embodiments of (i) option A, (ii) option B, and (iii) option A plus option B.

Where a numerical range is provided herein, it is understood that all numerical subsets of that range, and all the individual integers contained therein, are provided as part of the invention.

All combinations of the various elements described herein, including all subsets, are within the scope of the invention unless otherwise indicated herein or otherwise clearly contradicted by context.

The present invention will be further illustrated in the following Examples, which are given for illustration purposes only and are not intended to limit the invention in any way.

### Experimental Details

### Methods and Materials

Animals. Male C57BL/6 mice were obtained from Jackson Laboratories (Bar Harbor, ME) at 6-8 weeks of age. All mice were maintained in temperature-controlled rooms on a 12 h light-dark cycle with access to food and water ad libitum. All experimental protocols were approved by the Institutional Animal Care and Use Committee (IACUC) at the Feinstein Institute for Medical Research (FIMR), Northwell Health, which follows the NIH guidelines for ethical treatment of animals.

Production of recombinant ChAT protein. Production of recombinant ChAT protein. Recombinant human ChAT corresponding to residue 119-748 of respective protein (EC2.3.1.6) with a N-histidine tag was expressed in E. coli BL21 (Gold) DE3 cells. When bacteria were cultured to an A600 of 0.9, IPTG (isopropyl-1-thio-beta-D-galactopyranoside) was added to a final concentration of 3 mM to induce recombinant ChAT production. Bacteria were harvested and re-suspended in cold binding buffer and sonicated at 4° C. Cells debris was removed by centrifugation and the supernatant was applied onto a high affinity Ni-charged column pre-equilibrated with binding buffer. Following sequential washings, the recombinant histidine-tagged ChAT protein was eluted with 0.5 M imidazole, 10% glycerol, 20% IX DPBS. The recombinant ChAT was further purified by dialysis at 4° C. and extensive Triton X-114 extraction to remove contaminating endotoxins.

To increase the solubility and half-life of recombinant ChAT, unbranched amine-reactive MS (PEG) 12 reagent (#22685, Thermo Scientific) was used to achieve the ChAT PEGylation. The reaction was performed in a buffer containing 10% glycerol, 20% DPBS IX, 0.5 mM TCEP at a molar ratio of ChAT protein to MS (PEG) 12 of 1 to 200-fold molar excess for 20 h at 4° C. Following PEGylation, the PEGylated protein was then dialyzed in buffer and further extracted with Triton X-1 14 to remove contaminating endotoxins.

ChAT activity assay. Activity of recombinant and PEGylated recombinant ChAT were analyzed using a colorimetric assay. ChAT and its substrates choline and acetyl-coenzyme A are incubated at 37° C. for 15 minutes. After incubation, a cocktail containing choline oxidase, 4-aminoantipyrine, phenol, and HRP is added. A red color develops in proportion to the choline remaining in the reaction mixture. By subtracting the remaining choline in wells containing ChAT from the choline remaining in a well without any enzyme, the reaction rate can be calculated.

Data Analysis. Data were analyzed using Graphpad Prism 7.0. Statistical significance was calculated using one-way ANOVA with Tukey's correction for multiple comparisons or with paired t-tests. In all figures, * p<0.05; ** p<0.01; *** p<0.001; **** p<0.0001.

### Experiment 1: Endotoxemia and ChAT Treatment

### Methods

Endotoxemia was induced by injecting C57BL/6 mice intraperitoneally (i.p.) with 0.3 mg/kg endotoxin. Mice were treated intraperitoneally with the indicated dose of ChAT or PEG-ChAT or vehicle 30 min prior to endotoxin administration. Animals were euthanized by CO₂ asphyxiation 1.5 h after endotoxin injection, and blood was collected by cardiac puncture. Blood was centrifuged at 1,500 g for 15 min to isolate serum. Sera were used for TNF analysis by ELISA (R&D Systems) according to the manufacturer's recommendations.

### Results

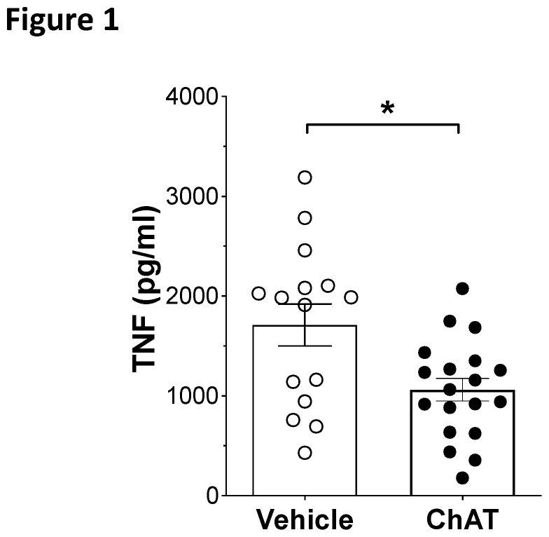

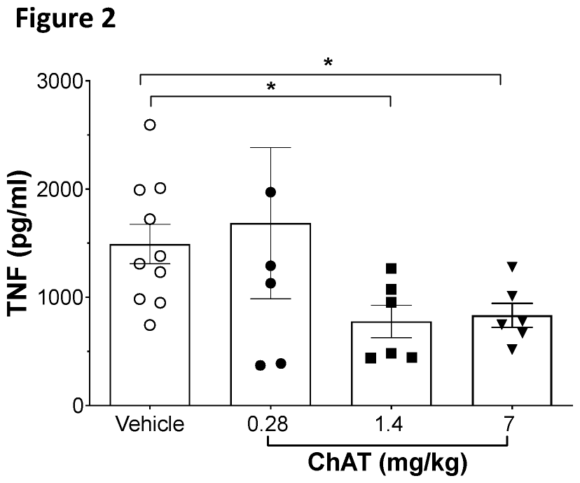

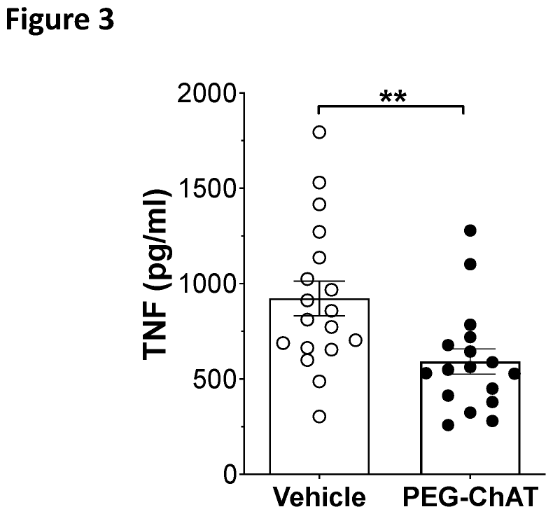

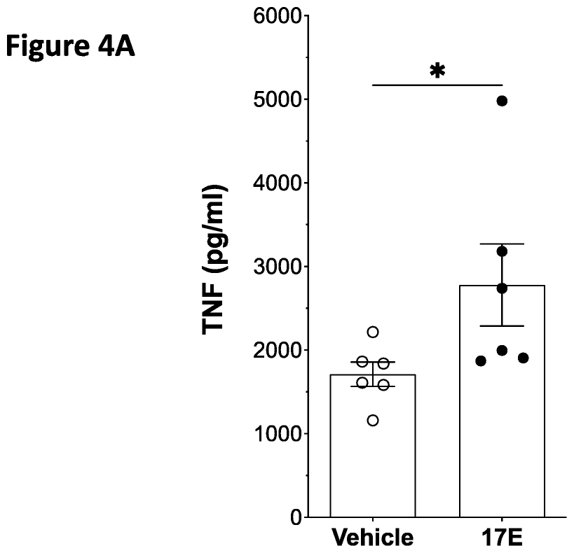

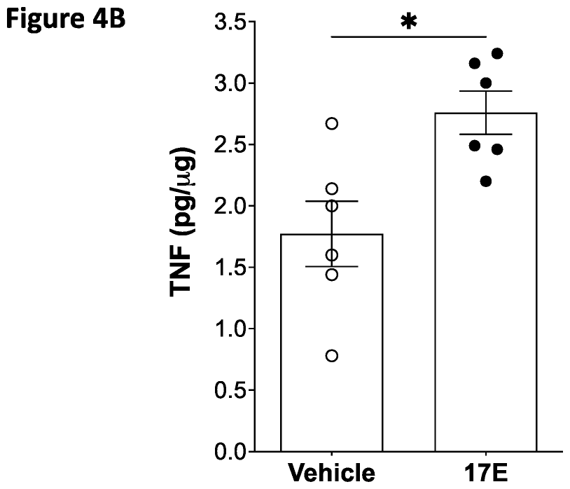

Administration of ChAT protein attenuated inflammation (measured as circulating TNF level) in response to the endotoxin challenge (FIG. 1). Moreover, as can be seen in FIG. 2, administration of ChAT protein reduced inflammation (measured as circulating TNF level) in response to an endotoxin challenge in a dose-dependent manner. FIG. 3 illustrates that PEG-ChAT was also effective in reducing inflammation (measured as circulating TNF level) in response to the endotoxin challenge. FIG. 4A-4B demonstrates that administration of an anti-ChAT antibody (17E) exacerbated TNF production in acute endotoxemia.

These results indicate that the intraperitoneal administration of ChAT or PEG-ChAT inhibited TNF levels in acute endotoxemia. Based upon this murine model, which is predictive for the in vivo treatment of inflammatory conditions in human and non-human animals, these results indicate that administering ChAT or PEG-ChAT intravenously or intraperitoneally would be an effective therapeutic to treat inflammatory conditions.

### Experiment 2: Cecal Ligation and Puncture Surgery and ChAT Treatment

### Methods

Severe polymicrobial sepsis was induced by cecal ligation and puncture (CLP). Mice were anesthetized using ketamine (100 mg/kg) and xylazine (8 mg/kg) administered intramuscularly. Abdominal access was gained via a midline incision. The cecum was isolated and ligated with a 6-0 silk ligature below the ileocecal valve and then punctured once with a 22 G needle. Stool (approximately 1 mm) was extruded from the hole, and the cecum placed back into the abdominal cavity. The abdomen was closed with two layers of 6-0 Ethilon sutures. An antibiotic (Imipenem-Cilastatin, 0.5 mg/kg, subcutaneously, in a total volume of 0.5 mL/mouse) was administered immediately after CLP as part of the resuscitation fluid. For survival studies, mice were randomized and injected intraperitoneally with ChAT or PEG-ChAT or vehicle once per day for 5 days. Survival was monitored for 2 wks.

### Results

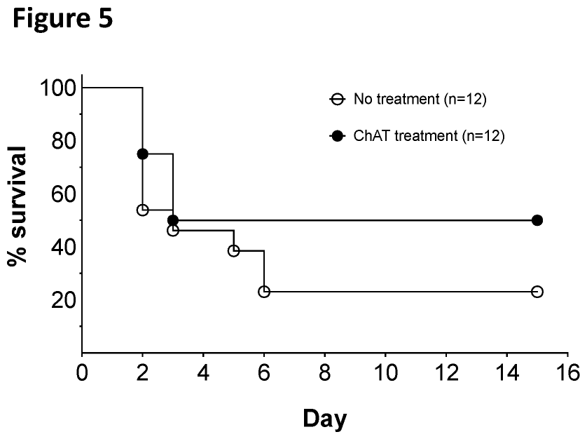

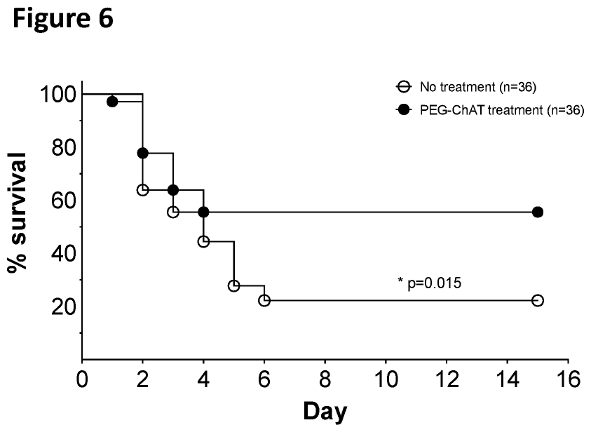

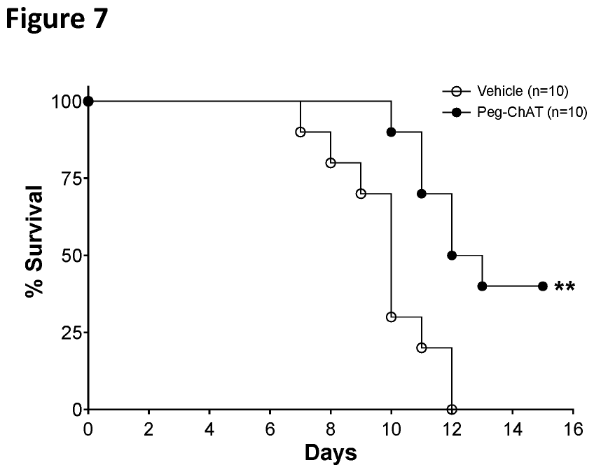

The intraperitoneal administration of ChAT protein (FIG. 5) or PEG-ChAT (FIGS. 6 and 7) improved the survival of the mice in the lethal sepsis model when compared to control. Based upon this murine model, which is predictive for the in vivo treatment of inflammatory conditions in human and non-human animals, these results indicate that administering ChAT or PEG-ChAT intravenously or intraperitoneally would be an effective therapeutic to treat sepsis.

### Experiment 3: Spike Protein-Induced Cytokine Response and ChAT Treatment

### Methods

C57BL/6 mice were injected intraperitoneally (i.p.) with SARS COV2-spike protein receptor binding domain (RBD). Mice were treated intraperitoneally with ChAT or PEG-ChAT or vehicle at 30 min prior to RBD administration. Animals were euthanized by CO₂ asphyxiation 2 h after RBD injection, and blood was collected by cardiac puncture. Blood was centrifuged at 1,500 g for 15 min to isolate serum. Sera were used for cytokine analysis using MSD assay according to the manufacturer's recommendations.

### Results

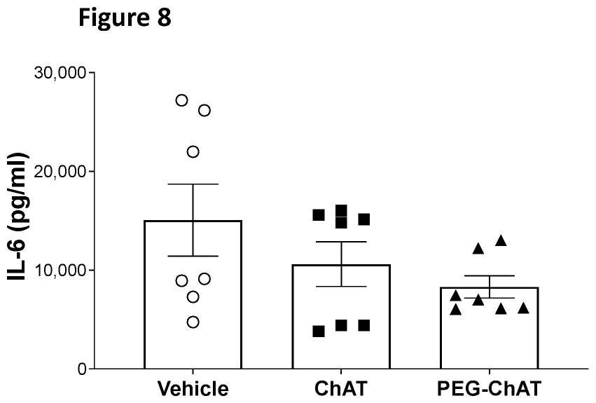

Administration of ChAT or PEG-ChAT protein attenuated inflammation (measured as circulating IL-6 level) in response to the SARS-COV2 receptor binding domain (RBD) challenge (FIG. 8). These results indicate that the intraperitoneal administration of ChAT or PEG-ChAT inhibited IL-6 levels. Extensive studies have shown that infection with SARS-CoC2 or similar viruses results in inflammatory conditions leading to cytokine storm and associated pathologic conditions. These results indicate that administering ChAT or PEG-ChAT intravenously or intraperitoneally would be an effective therapeutic to treat inflammatory conditions after SARS-COV2 or other similar pathogen infections (viral, bacterial, microbial, fungal, parasitic).

### Experiment 4: Colitis and ChAT Treatment

### Methods

DSS colitis was induced in 8-week-old wild-type C57BL/6 mice by the addition of 4% DSS (M.W 36 000-50 000) in drinking water for 7 days. Mice were treated intraperitoneally with ChAT or PEG-ChAT or vehicle twice per day for 7 days, starting on Day 8. Body weight of the animals was monitored every day during the experimental period. Cheek bleed samples were collected on days 0, 7 and 14. Blood was centrifuged at 1,500 g for 15 min to isolate serum. Sera were used for cytokine analysis using MSD assay according to the manufacturer's recommendations. At the end of the experiment, animals were euthanized by CO₂ asphyxiation, and colons were harvested, and measured in length. Disease activity index, was derived from body weight loss and change in colon length/weight.

### Results

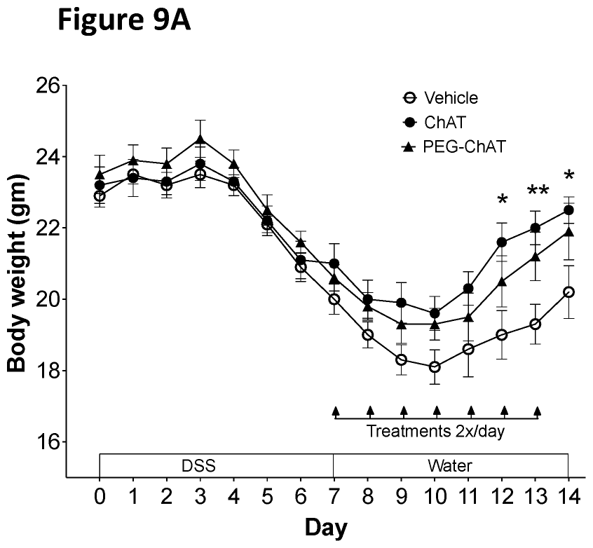

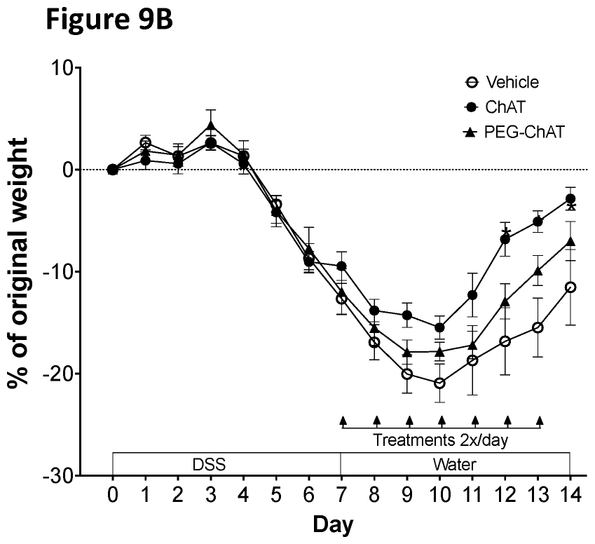

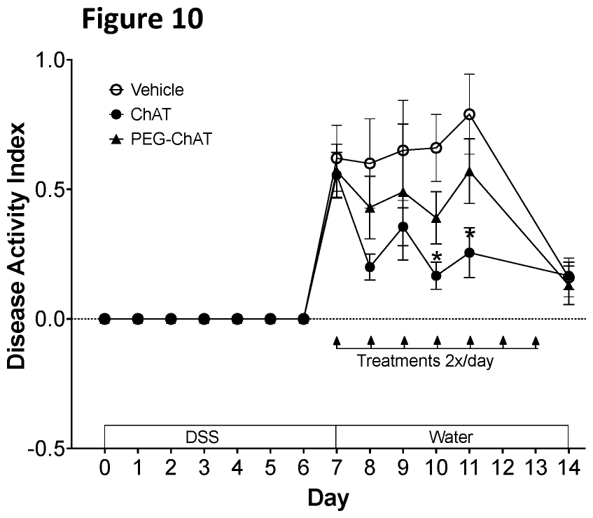

FIG. 9A and FIG. 9B illustrate that following intraperitoneal administration of either ChAT or PEG-ChAT, experimental animals showed less loss in body weight when compared to control. Further, both ChAT and PEG-ChAT showed an improved disease activity index when compared to control in another preclinical model of colitis (FIG. 10).

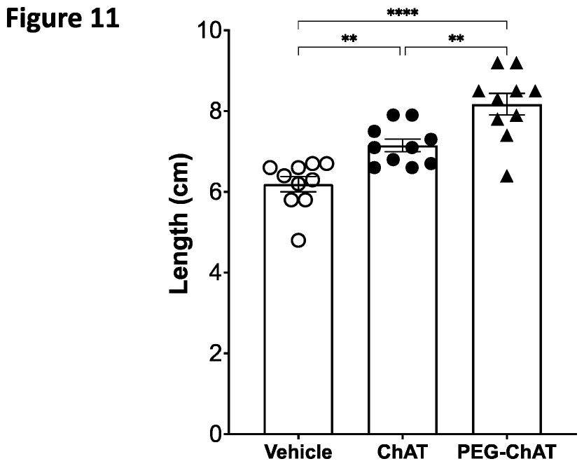

FIG. 11 summarizes the colon length taken from mice in a control group, mice treated with ChAT and mice treated with PEG-ChAT. As can be seen, the colons were longer in mice that were administered ChAT or PEG-ChAT.

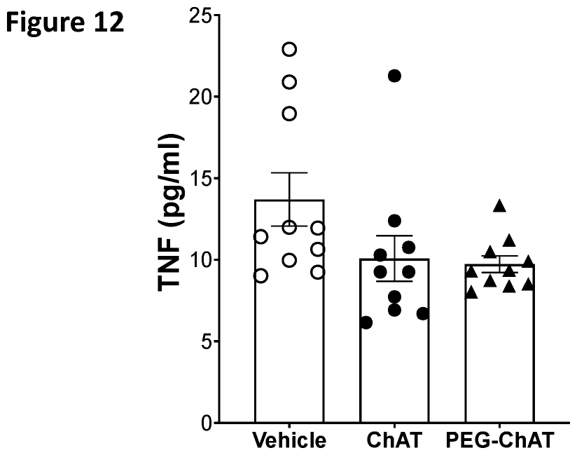

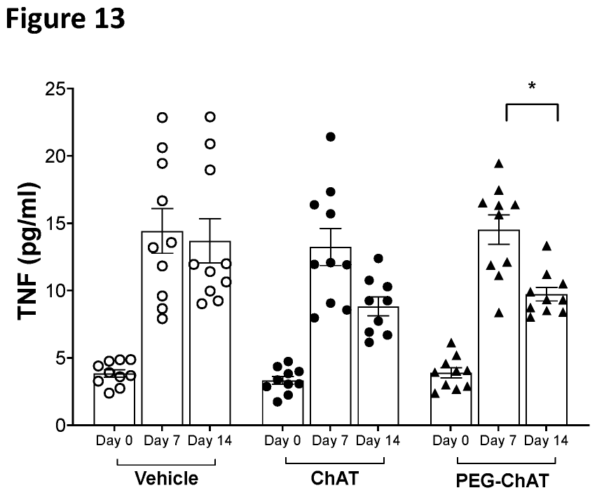

FIG. 12 and FIG. 13 illustrate that both ChAT and PEG-ChAT when administered intraperitoneally significantly decreased TNF levels, indicating that both ChAT and PEG-ChAT would be effective in treating colitis.

Taken together, these results demonstrate that both ChAT and PEG-ChAT were effective in multiple preclinical models of colitis. Since these models are recognized in the art to be predictive of treating this condition in humans and non-human animals, it is reasonable to conclude that administering ChAT or PEG-ChAT intravenously or intraperitoneally or by rectal administration would be an effective therapeutic to treat colitis in human and non-human animals.

### Experiment 5

### Methods

Wild-type C57BL/6 mice received ChAT, pegylated ChAT (PEG-ChAT) or vehicle intra-peritoneally 30 minutes before 0.3 mg/kg lipopolysaccharide (LPS). 90 minutes after LPS administration, mice were euthanized, and serum and spleen samples were collected. Tumor necrosis factor (TNF) levels were measured using ELISA. Wild-type C57BL/6 mice were subjected to a cecal ligation and puncture (CLP)-induced peritonitis. ChAT, PEG-ChAT or vehicle were administered intraperitoneally once per day for 5 consecutive days. Survival was observed for 15 days post-CLP. DSS colitis was induced in 8-week-old wild-type C57BL/6 mice on Day 0 by the addition of 4% DSS (M.W 36 000-50 000) in drinking water for 7 days. Mice received ChAT, PEG-ChAT or vehicle intraperitoneally twice per day for 7 days, starting on Day 8.

### Results

Administration of ChAT or PEG-ChAT significantly inhibits serum TNF levels (p=0.002) in response to endotoxin challenge as compared to vehicle controls (ChAT versus vehicle: 1062.0 pg/mL+113.0, n=19 versus 1711.0 pg/mL+210.2, n=15, and PEG-ChAT versus vehicle: 592.3 pg/mL+65.95, n=17 versus 921.9 pg/mL+90.59, n=18). In a murine sepsis model, administration of PEG-ChAT significantly improved survival (p=0.015), with a 55.6% survival in ChAT treated mice as compared to a 22.2% survival in vehicle-treated mice. In DSS-induced colitis model, ChAT administration significantly improved body weight gain (d14 p=0.01 ChAT mice: −5.1%±1.1, n=10 versus vehicle mice: −15.5%±2.9, n=10), disease score (d11 p=0.006 ChAT mice: 0.17DAI±0.05, n=9 versus vehicle mice: 0.66DAI±0.13, n=10), and colon length (p=0.006 ChAT mice: 7.15 cm±0.2, n=10 versus vehicle mice: 6.2 cm±0.2, n=10) compared to vehicle.

### Experiment 6—Combination Dexamethasone+ChAT or PEG-ChAT Administration in Acute Endotoxemia

### Methods

Using C57BL/6 male mice, 7-8 weeks of age, dexamethasone (0.25 mg/kg) and ChAT (1 mg/kg) or PEG-ChAT (1 mg/kg) were administered i.p., followed 30 minutes later by LPS (0.3 mg/kg) i.p. The animals were sacrificed 90 minutes later.

### Results

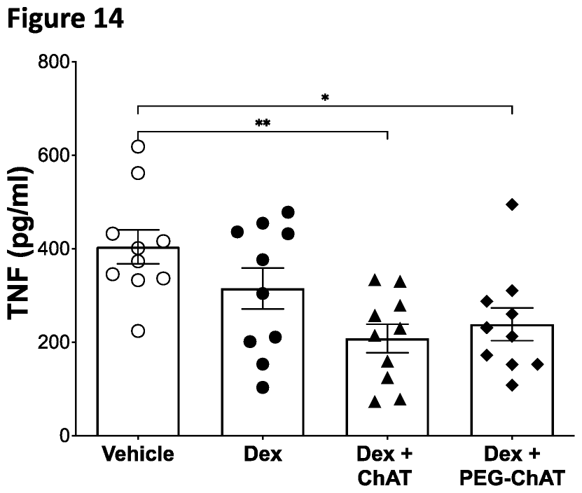

Administration of either ChAT or PEG-ChAT in combination with dexamethasone was effective in reducing TNF levels (FIG. 14).

These results indicated that administration of ChAT inhibited TNF levels in acute endotoxemia and attenuated disease severity in murine models of sepsis and DSS-induced colitis, which are recognized in the art to be predictive of treating these disease state, and other inflammatory conditions that exhibit profiles of increased inflammatory/pro-inflammatory cytokines, in vivo. These results indicate that administering ChAT or PEG-ChAT intravenously or intraperitoneally would be an effective therapeutic to treat inflammatory conditions and sepsis in humans and non-human animals.

Having thus described in detail preferred embodiments of the present invention, it is to be understood that the invention defined above is not limited to particular details set forth in the above description as many apparent variations thereof are possible without departing from the spirit or scope of the present invention. All publications cited herein and publications referenced in those documents (including manufacturer's instructions, descriptions and product specifications), are expressly incorporated herein by reference in their entirety and may be employed in the practice of the invention.

1. A method of treating one or more of endotoxemia, sepsis, colitis, an inflammatory condition, and symptoms associated with COVID-19 in a subject in need thereof, which comprises administering an effective amount of a composition comprising choline acetyl transferase (ChAT) or ChAT conjugated to polyethylene glycol (PEGylated ChAT) and a pharmaceutically acceptable carrier to said subject.

2. The method of claim 1, wherein the subject is a human.

3. The method of claim 1, wherein the subject is a non-human animal.

4. The method of claim 1, wherein the composition comprises ChAT and a pharmaceutically acceptable carrier.

5. The method of claim 1, wherein the composition comprises PEGylated ChAT.

6. The method of claim 5, wherein the PEGylated ChAT is ChAT conjugated to about 2 to about 24 polyethylene glycol chains and wherein each polyethylene glycol chain has a molecular weight of about 200 to about 2,000 daltons.

7-8. (canceled)

9. The method of claim 1, wherein the subject has sepsis.

10. The method of claim 1, wherein the subject has endotoxemia.

11. The method of claim 1, wherein the composition further comprises an acetylcholine esterase inhibitor.

12. The method of claim 11, wherein the acetylcholine esterase inhibitor is neostigmine, donepezil, rivastigmine, or galantamine.

13. The method of claim 1, wherein the composition further comprises choline, acetyl coenzyme A, or both.

14. The method of claim 1, wherein the subject has colitis.

15. The method of claim 1, wherein the subject has an inflammatory condition.

16-22. (canceled)

23. The method of claim 15, wherein the inflammatory condition is myocarditis, pancreatitis, inflammatory bowel disease, rheumatoid arthritis, osteoarthritis, or asthma, or wherein the inflammatory condition is due to chemotherapy-induced toxicity or to radiation-induced toxicity.

24-26. (canceled)

27. The method of claim 1, wherein the composition further comprises an anti-inflammatory agent.

28. The method of claim 27, wherein the anti-inflammatory agent is a non-steroidal anti-inflammatory agent.

29. The method of claim 28, wherein the non-steroidal anti-inflammatory agent is aspirin, indomethacin, naproxen sodium, celecoxib, diclofenac, diflunisal, etodolac, ketoprofen, ibuprofen, fenoprofen, meloxicam, piroxicam, tolmetin, sulindac, phenylbutazone, or oxyphenbutazone,

30. The method of claim 27, wherein the anti-inflammatory agent is a steroid.

31. The method according to claim 30, wherein the steroid is prednisone, triamcinolone, hydrocortisone, beclomethasone, betamethasone, cortisone, or dexamethasone.

32. (canceled)

33. The method of claim 1, wherein administering the composition comprising choline acetyl transferase (ChAT) or ChAT conjugated to polyethylene glycol (PEGylated ChAT) and a pharmaceutically acceptable carrier to the subject reduces circulating inflammatory cytokine levels in the subject.

34-48. (canceled)

49. The method of claim 1, wherein the subject has symptoms associated with COVID-19.

50-52. (canceled)


SEQUENCE LISTING (2 sequences)

SEQ ID NO: 1 - protein, 748 aa, Homo sapiens
MGLRTAKKRGLGGGGKWKREEGGGTRGRREVRPACFLQSGGRGDPGDVGGPAGNPGCSPHPRAATRPPPLPAHTPAHTPEWCGAASAEAAEPRRAGPHLCIPAPGLTKTPILEKVPRKMAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKPELSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDGPGGVELSDTHRALQLLHGGGYSKNGANRWYDKSLQFVVGRDATCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 2 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP